(19) United States
(12) Patent Application Publication
(10) Pub. No.: US 2024/0335649 A1
(43) Pub. Date: Oct. 10, 2024

(54) DEVICE AND METHOD TO IMPROVE CARDIAC CONTRACTILITY AND ENABLE CARDIAC RECOVERY IN PATIENTS WITH HEART FAILURE

(71) Applicants: Kalyanaraman ANANTHANARAYANAN, TAMIL NADU (IN); DHEERAJ REDDY, TAMIL NADU (IN)

(72) Inventors: Kalyanaraman ANANTHANARAYANAN, TAMIL NADU (IN); DHEERAJ REDDY, TAMIL NADU (IN)

(21) Appl. No.: 18/380,206

(22) PCT Filed: Apr. 18, 2022
(86) PCT No.: PCT/IB2022/053605

§ 371 Date: Oct. 16, 2023

(30) Foreign Application Priority Data
Apr. 19, 2021 (IN) 202141018098

(51) Int. Cl. A61M 60/523; A61M 31/00; A61M 60/178; A61M 60/232; A61M 60/237; A61M 60/258; A61M 60/405; A61M 60/427; A61M 60/531; A61M 60/873; A61M 60/876; A61N 1/05; A61N 1/39

(52) U.S. Cl. CPC A61M 60/523; A61M 31/002; A61M 60/178; A61M 60/232; A61M 60/237; A61M 60/258; A61M 60/405; A61M 60/427; A61M 60/531; A61M 60/873; A61M 60/876; A61N 1/0587; A61N 1/3962; A61M 2205/054; A61M 2205/3523; A61M 2230/04

(57) ABSTRACT

The present invention helps in enabling cardiac recovery in patients with heart failure and for facilitating cardiac muscle regeneration by long term augmentation of the coronary perfusion pressure and thereby the coronary blood flow by using an implantable, preferably self-contained electrically operated pump which takes the oxygen rich blood from one of the chambers of the heart or a large blood vessel, that include the aorta, the subclavian artery, left atrium, one or more pulmonary veins, etc and pumps it into a singular or multitude of coronary arteries through a natural (autologous, homologous or heterologous) or synthetic ‘conduit’, a medium to long term inotropic support to the heart, through a dedicated channel to deliver inotropic and other medicines and stem cells directly into the coronary circulation, where one end of the channel typically ends outside the body and the other end rests inside the lumen of the ‘conduit’ which supplies the oxygenated blood into the coronary arteries; a central processing unit (CPU) with an integrated defibrillator to monitor and control pump speed and flow rates; an implantable power source for the pump which can be charged transcutaneously using an external wireless system (‘TET’: transcutaneous energy transfer systems), to power the pump and the CPU.

### TECHNICAL FIELD

This invention relates to a medical device, more specifically an implantable, self-contained device intended to support and enable recovery of cardiac muscle, which is meant to be used on a medium to long term basis in heart failure patients, thereby reducing their hospitalisation and duration of hospital stay, while permitting the patients to lead an active and productive life while still under therapy for heart failure. This device is designed to augment coronary perfusion pressure, improve cardiac contractility (both directly as well as indirectly) and promote heart muscle healing and regeneration, thereby avoiding the need for more morbid, riskier or permanent procedures like a Ventricular Assist Device (VAD) insertion or a heart transplant. For a very large fraction of heart failure patients this can be a bridge to recovery or a destination therapy, and in a small fraction of patients this can be used as a bridge to heart transplant or VAD.

### BACKGROUND OF THE INVENTION

Heart failure is one of the most common causes of morbidity and death across the world. Heart failure is generally a heart muscle weakness and inability to pump sufficient blood to meet the body's demands. Currently about 40 million people worldwide suffer from heart failure. This is about 2% of the total world population. If we look at the age wise incidence, about 6% of the population over 65 years of age and about 10% of the population over 75 years of age suffer from heart failure. With the world population ageing gradually, the number of patients with heart failure is likely to increase in the coming decades.

The most common causes of heart failure are ischaemic heart disease and cardiomyopathies. They cause permanent damage to the cardiac muscle (myocardium) and reduce its pumping ability. The pumping ability of the heart, more importantly the Left Ventricle, is assessed by its Ejection Fraction (LVEF). In normal hearts, the LVEF is about 60-70%. Symptoms of heart failure manifest if the LVEF decreases to 40% or less. They are more pronounced if the LVEF is less than 30%, and gradually progress to life threatening situation when the LVEF falls to below 25%.

In a smaller subgroup of patients with heart failure, the right ventricle may be affected, manifesting as right ventricular or biventricular failure. In the later stages of heart failure, biventricular failure ensues almost invariably.

Heart failure is classified into four stages based on gradually increasing symptoms (NYHA Classification). Patients having ejection fraction of less than 30% generally fall under NYHA Class III and patients with LVEF of about 20% generally have NYHA Class IV symptoms and these will be the patients requiring frequent hospitalisations, ICU admissions, prolonged hospital stay, and are candidates for long term circulatory support in the form of VAD or heart transplants. NYHA Class III failure manifests as shortness of breath, poor exercise tolerance, fatigue and weakness and other cardiac symptoms even during day-to-day activities, with or without medications. NYHA Class IV manifests as of breathlessness, palpitations and other cardiac symptoms even at rest. The patients having Stage III symptoms worsen rapidly to Stage IV in the absence of medical therapy and more gradually with medical therapy. This gradual worsening of the myocardial function is attributed to the gradual reduction of coronary perfusion pressure ‘CPP’ (The driving force of blood into the coronary arteries, defined as the difference between diastolic blood pressure (DBP) in the aorta and the (end) diastolic pressure inside the Left Ventricular cavity (LVDP) of the heart) over a period of time. Patients with symptoms of Stage IV heart failure are generally candidates for heart transplants or mechanical support devices (VAD: Ventricular Assist Devices).

Heart is not a terminally differentiated organ as assumed till recent times. Recent studies in stem cell therapy for heart failure offer a ray of hope. Even though Stem cell therapy as a one-time standalone therapy may not have been proven to be very effective, direct intracoronary injection of stem cells with other growth stimulators into the heart several times over a span of weeks or months when coupled with continuous coronary perfusion pressure (CPP) augmentation, has a very strong possibility of regenerating the cardiomyocytes.

Other mechanical assist devices used to manage heart failure patients like Ventricular Assist Devices (VADs) use electrically driven pumps to remove blood from the left ventricle ‘LVAD’ (or right ventricle, in case of Right Ventricular Assist Device, ‘RVAD’) and pump it into the aorta (or pulmonary artery, in case of RVAD). These pumps require a lot of electrical energy that they have a power line protruding out of the patient's body, and the pump needs to be constantly powered by a battery pack, carried by the patient all the time. The ventricular assist devices (VAD) focus on improving the cardiac output and offloading the ventricle. They don't focus on augmenting the coronary perfusion pressure (CPP) and they don't allow direct injection of cardiac stimulating medications and stem cells into the coronary arteries enabling cardiac muscle recovery.

Of the other mechanical assist devices, notably the Intra-Aortic Balloon Pump (IABP), uses a helium gas inflating system coupled with a balloon which is positioned in the aorta, which gets inflated and deflated alternatively during diastole and systole, to augment coronary perfusion pressure. But this device system works only on a short term (days to weeks) in patients confined to a hospital bed in an ICU, and requires round the clock intensive monitoring by healthcare personnel. They cannot be used in out of hospital settings and in ambulant patients, who lead a productive life.

Cardiac stimulant medicines and coronary dilators like adrenaline, milrinone, dobutamine, nitroglycerine, levosimendan, nicorandil etc, are conventionally delivered to the heart through the blood stream through a venous catheter. The side effects of these medicines are enormous in this method of usage, as the dose of these abovementioned medicines will increase by 15-20 times if they are delivered intravenously instead of administering them through the intracoronary route. In these concentrations, adrenaline causes peripheral vasoconstriction, increase cardiac workload, cause cardiac arrhythmias and ischemia of vital organs. Milrinone, Levosimendan and Nicorandil cause hypotension and reduced coronary perfusion.

One of the prior art means is disclosed in An article named as “A Real-Time Health Monitoring System for Remote Cardiac Patients Using Smartphone and Wearable Sensors” is published in “International Journal of telemedicine and applications” by Priyanka Kakria, N. K. Tripathi, and Peerapong Kitipawang

In this study a real-time heart monitoring system for heart patients located in remote areas has been proposed. The developed system is comprised of wearable sensors, Android handheld device, and web interface. The system is adaptable and has the ability to extract several cardiac parameters such as heart rate, blood pressure, and temperature of multiple patients simultaneously. The extracted data is being transmitted to Android handheld device using Bluetooth low energy which is then transmitted to web application for further processing. Web application processes received data to show medical status of the patient along with personal information such as age, gender, address, and location on web interface. An alarming system based on threshold values has also been designed which sends alert message to the doctor in case of abnormalities such as arrhythmia, hypotension, hypertension, fever, and hypothermia.

Another prior art means is disclosed in “An Energy Efficient Wearable Smart IOT System to Predict Cardiac Arrest” published in Advances in Human computer interaction in 2019. The objective of this research is to present a multisensory system using a smart IOT system that can collect Body Area Sensor (BAS) data to provide early warning of an impending cardiac arrest. The goal is to design and develop an integrated smart IOT system with a low power communication module to discreetly collect heart rates and body temperatures using a smartphone without it impeding on everyday life. This research introduces the use of signal processing and machine-learning techniques for sensor data analytics to identify predict and/or sudden cardiac arrests with a high accuracy. The IOT device constantly collects data from the user and sends it to smartphone via a Bluetooth communication module. All the processing and data analysis take place in the application where the user has the option to view user real-time plots. These plots provide the user a basic idea of his/her body's status. The user does not have maintained a record of his/her data to ensure that s/he is in a healthy or unhealthy state since the application's job is to alert the user upon an emergency. Finally, when the algorithm senses an abnormality it immediately alerts the user.

Another prior art means is disclosed in U.S. Pat. No. 7,647,102 titled as a Cardiac contractility modulation device having anti-arrhythmic capabilities and method of operating thereof. A cardiac contractility modulating (CCM) device (30) includes an anti-arrhythmic therapy unit (38) for detecting a cardiac arrhythmia in a heart (2) of a patient based on processing electrical signals related to cardiac activity sensed at the heart, and for delivering anti-arrhythmic therapy to the heart. The device includes a cardiac contractility modulating (CCM) unit (40) capable of delivering cardiac contractility modulating (CCM) signals to the heart for modulating the contractility of a portion of the heart. The device may provide to the anti-arrhythmic therapy unit control signals associated with the delivery of the CCM signals to the heart. The control signals may be used to prevent interference of the CCM signals with the detecting of the cardiac arrhythmia. The device (30) includes a power source. The device may be an implantable device or a non-implantable device. The device may also include a pacing unit.

The prior art devices do not show the use in-built sim card model to transmit vital data including the pump speed, coronary artery pressure, heart rate, percentage of power remaining in the battery, etc. to the smart phones.

So there is a need for a device to improve cardiac muscle contractility and enable cardiac recovery in patients with heart failure and to enable the patient to perform day-to-day activities without any discomfort. The device of the present invention minimizes the discomforts like breathlessness, palpitations and other cardiac symptoms and also minimizes the side effects of cardiac stimulant by administering through the intracoronary route. The device of the present invention helps in preventing the patients from frequent hospitalisations, ICU admissions and prolonged hospital stay. The device of the present invention also helps to administer the combination of cardio-stimulant through infusor pump.

### SUMMARY OF THE INVENTION

The present invention is a system for improving the contractility of cardiac muscle, for alleviating the symptoms of cardiac failure on a medium to long term basis and for facilitating cardiac muscle regeneration by long term augmentation of the coronary perfusion pressure and thereby the coronary blood flow by using an implantable, preferably self-contained electrically operated pump. This pump takes the oxygen rich blood from one of the chambers of the heart or a large blood vessel, that include the aorta, the subclavian artery, left atrium, one or more pulmonary veins etc, and pumps it into a singular or multitude of coronary arteries through a natural (autologous, homologous or heterologous) or synthetic ‘conduit’, to which the coronary arteries are anastomosed in a side-to-side or end-to-side fashion; a medium to long term inotropic support to the heart, through a dedicated channel to deliver inotropic and other medicines and stem cells directly into the coronary circulation, where one end of the channel typically ends outside the body of the human or animal and the other end rests inside the lumen of the ‘conduit’ which supplies the oxygenated blood into the coronary arteries; a central processing unit (CPU) with an integrated defibrillator to monitor and control pump speed and flow rates; an implantable power source for the pump which can be charged transcutaneously using an external wireless system (‘TET’: transcutaneous energy transfer systems), to power the pump and the CPU. This device uses electric energy to only improve coronary perfusion and uses physiological energy through inotrope infusion to improve cardiac output (as opposed to ventricular assist devices, which use electric energy to improve cardiac output), the electrical energy used by this device will be roughly one tenth of that of ventricular assist devices. Hence this device can be easily run on wireless charging and there will be no need for a power supply line extending through the patient's skin (percutaneous) which will need to be connected to battery packs round the clock. This design takes away the discomfort of carrying a heavy battery pack round the clock and eliminates the risk of driveline infection which has a very high incidence of life-threatening sepsis. Also, this design will allow the patients to engage in contact sports and swimming which were not possible for patients under ventricular assist device (VAD) support.

### BRIEF DESCRIPTION OF THE DRAWINGS

FIG. 1 Front view of the heart showing the coronary arteries. Coronary arteries which are not visible in the front view are shown in dotted lines.

FIG. 2 Heart viewed from behind: showing coronary arteries which are visible on the under surface of the heart.

FIG. 3 Picture of the preferred embodiment of the device disclosed and its components.

FIG. 4 Picture of the preferred embodiment of the disclosed device revealing the cut section of the electrically driven “mechanical pump assembly” which is housed in the tubular member.

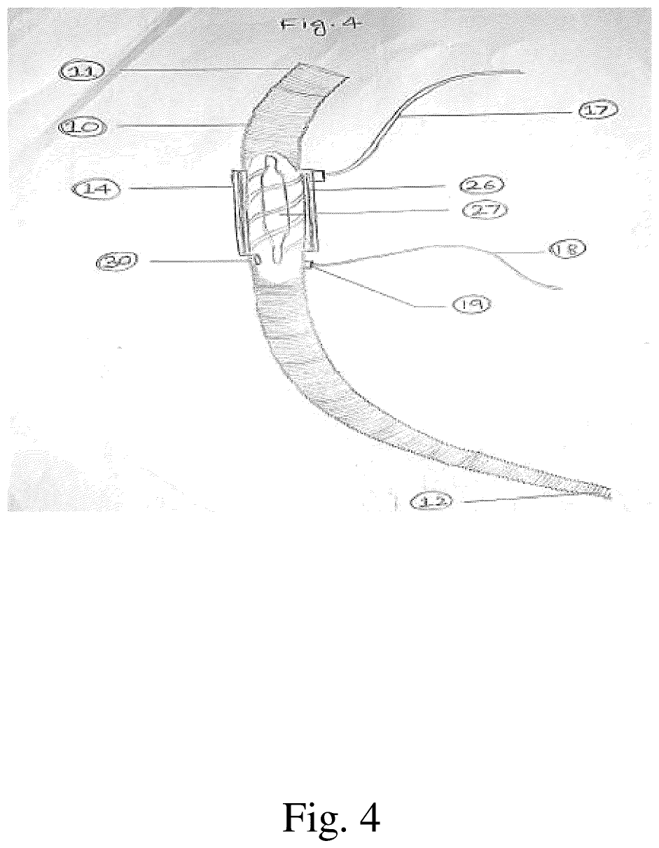

FIG. 5 The representation of the preferred embodiment of the device system as intended to be used in a human subject.

FIG. 6 The representation of a preferred method of anastomosing the tubular member to the vascular structures, namely end-to-side anastomoses at the aorta (proximal end) and pulmonary artery (distal end), and side-to-side anastomoses on multiple coronary arteries (distal portion of the tubular member).

FIG. 7 The representation of another method of anastomosing the tubular member to the vascular structures, namely end-to-side anastomoses at the Left Subclavian artery (proximal end) and pulmonary artery (distal end), and side-to-side anastomoses on multiple coronary arteries (distal portion of the tubular member).

FIG. 8 The representation of yet another method of anastomosing the tubular member to the vascular structures, namely end-to-side anastomoses at the Left atrial appendage (proximal end) and aorta (distal end), and side-to-side anastomoses on multiple coronary arteries (distal portion of the tubular member)

FIG. 9 Alternate embodiment of the distal portion of the tubular member giving out multiple smaller branches (‘distribution channels’) at its distal end, enabling end-to-side anastomoses with multiple coronary arteries.

FIG. 10 Comparison of the relation between Systolic Blood Pressure (SBP), Diastolic Blood Pressure (DBP), Left Ventricular Diastolic Pressure (LVDP) and Coronary Perfusion Pressure (CPP) with diastolic duration, in (A) Normal Heart, (B) Failing Heart, and (C) Failing heart with the support of the disclosed device.

FIG. 11 Transcutaneous Energy Transfer (TET): Charging of the implanted battery power source, by placing transmitter coils across the skin, directly over the subcutaneously implanted receiver coils.

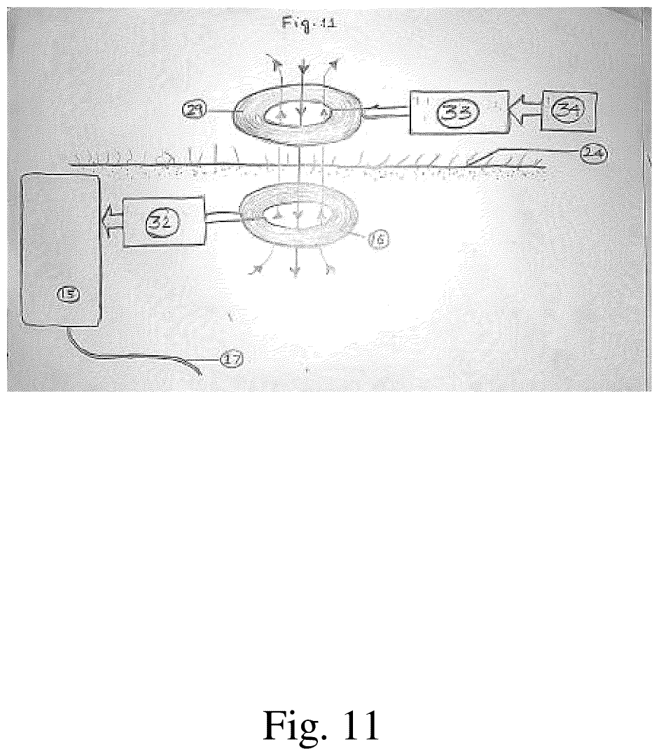

### DETAILED DESCRIPTION OF THE PREFERRED EMBODIMENT

Heart failure requires frequent hospitalisations to treat acute decompensations and life-threatening episodes. Hence the cardiac support in patients with heart failure is broadly classified into immediate support (days to weeks), short term support (weeks to months), intermediate term support (months to years) and long-term support (years to decades). IABP (Intra-Aortic Balloon pump is an example of immediate cardiac support, ECMO is an example of short-term cardiac support, LVADs and Heart transplants are examples of long-term cardiac support. Our invention is designed to provide intermediate (by coronary infusion of cardiostimulants) to long term support (by augmenting the coronary perfusion pressure) to heart and aid its recovery.

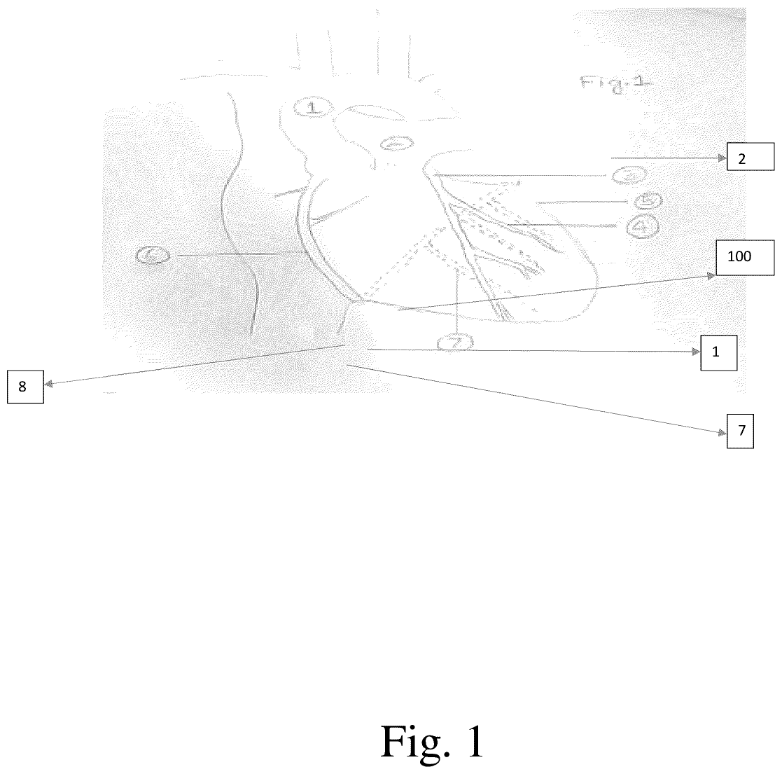

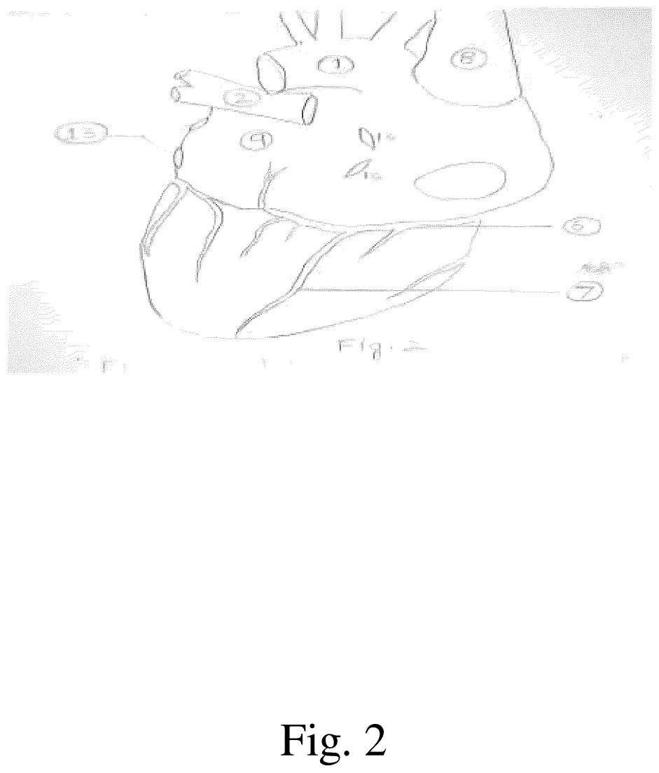

The blood supply of the heart is through the coronary arteries, which are illustrated in FIG. 1 and FIG. 2. FIG. 1 depicts the heart and the coronary arteries as seen from the front, the FIG. 2 depicts the heart and the coronary arteries as seen from behind. Normally about 300 ml of oxygen rich blood flows into the heart through the coronary arteries.

Of the cardiac chambers, the ventricles, more so the left ventricle which pumps blood to all parts of the body, is most dependent on the coronary arteries for its blood supply. The coronary blood flow to the left ventricle is mainly through the three coronary arteries, the Left anterior descending artery (3), the obtuse marginal branches (5) of the left circumflex artery, and the posterior descending branch (7) of the right coronary artery (6). The left anterior descending artery (3), supplies the anterior wall and the apex of the left ventricle through the diagonal branch (4).

The left anterior descending artery (3), along with its diagonal branch (4), supplies about 65% of the blood supply to the left ventricle. The remaining 35% blood supply is through the obtuse marginal branches (5), and the posterior descending branch (7) of the right coronary artery (6).

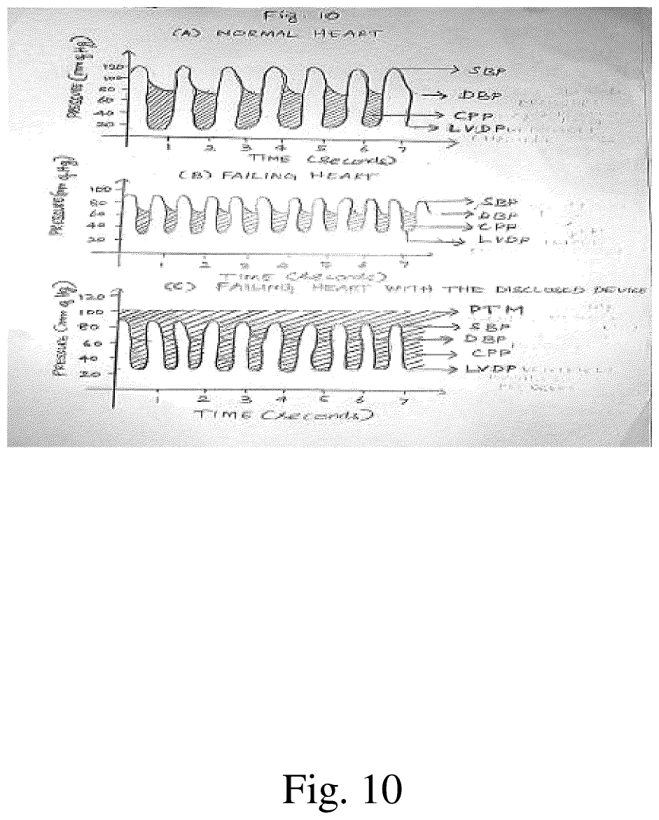

FIG. 10 illustrates the role of coronary perfusion pressure in maintaining coronary circulation. The graphs (A), (B) and (C) shows the relationship between systolic blood pressure (SBP), (which is the same in the LV chamber as well as in the arterial system), diastolic blood pressure (DBP) in the arterial system, left ventricular diastolic pressure (LVDP) and the coronary perfusion pressure (CPP). The cardiac tissue in the left ventricle gets its blood supply only during diastole. The amount of blood flowing through the coronary arteries is directly proportional to the coronary perfusion pressure (CPP) and the duration of diastole (normal: about 500 milliseconds). Coronary perfusion pressure (CPP) is the difference between the diastolic blood pressure (DBP) and the left ventricular diastolic pressure (LVDP). In the failing heart, gradually the LVDP keeps rising and the DBP keeps decreasing over weeks to months, thereby seriously compromising on the CPP. Moreover, since the duration of systole is constant, with increasing heart rate, as happens during heart failure, the diastolic duration keeps reducing to about 200 milliseconds or less, which along with the falling CPP, severely compromises the cardiac blood supply, resulting in a vicious cycle of accelerated worsening of cardiac failure and results in death. In FIG. 10(A), the shaded area shows the product of coronary perfusion pressure and the duration of diastole (CPP×duration) in the normal heart. In FIG. 10(B) clearly sees the reduction in shaded area, as happens with the fall in coronary perfusion in a failing heart, due to both a reduction in CPP as well as reduction in the duration of diastole. FIG. 10(C) shows the improvement in the coronary perfusion achieved by maintaining suprasystemic blood pressures in the tubular member (10) (Pressure in Tubular Member, ‘PTM’ in FIG. 10), despite the low duration of diastole (about 200 milliseconds), as disclosed in the following paragraphs. Essentially, the coronary perfusion pressure (CPP) in patients with heart failure who have this device implanted, is the difference between PTM and LVDP (even after ignoring the coronary perfusion which happens during the systolic phase of the cardiac cycle), which helps their hearts to achieve much higher coronary perfusion and aids in cardiac muscle recovery.

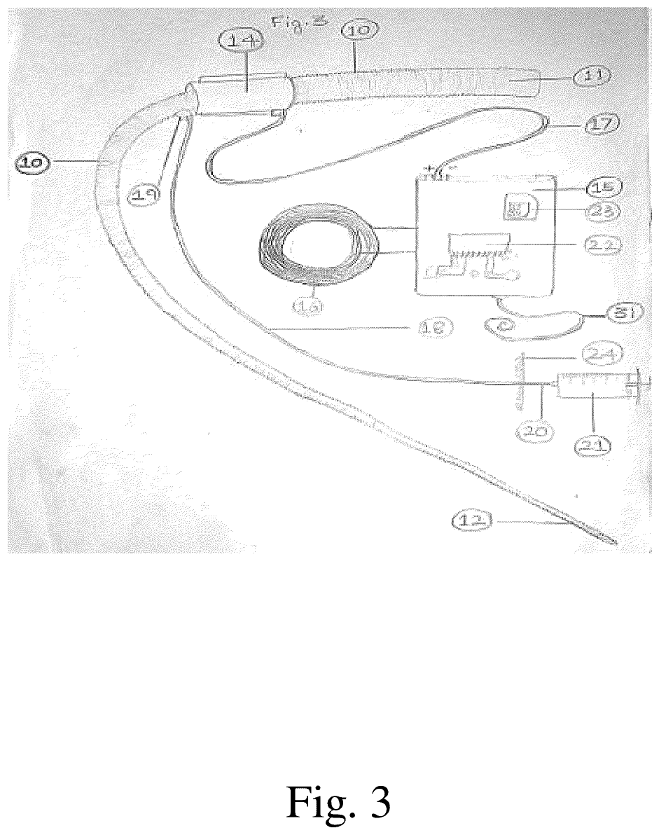

As illustrated in FIG. 3, the device includes a flexible, hollow tubular member body (10) made of autologous, homologous, heterologous, cell culture, synthetic material which includes dacron, teflon etc, or a combination of materials. It is impregnated or lined with a material to prevent tissue reactivity/blood-clot formation. Other examples of the material in which the tubular member is made of human, porcine, bovine arterial or venous conduits alone or in combination with other organic and/or synthetic materials. The tubular member body (10) has a proximal end (11) and a distal end (12). Since the proximal end (11) and the distal end (12) are defined, the rest of the ‘tubular member body’ is understood to be its ‘body’ and hence the terms ‘tubular member body’, “tubular member’ or ‘body’, is used interchangeably, unless specified otherwise. The tubular member body (10) houses an electrically driven ‘mechanical pump assembly’ (14) which propels the blood through the tubular member towards its distal end (12), generating suprasystemic pressures, at least during diastole. The tubular member body (10) is shaped in such a manner that it is wider (5-15 mm in diameter) at the proximal end (11) and narrower (about 5 mm diameter or less) at the distal end (12), and the tapering is in a uniform manner or in a stepped manner.

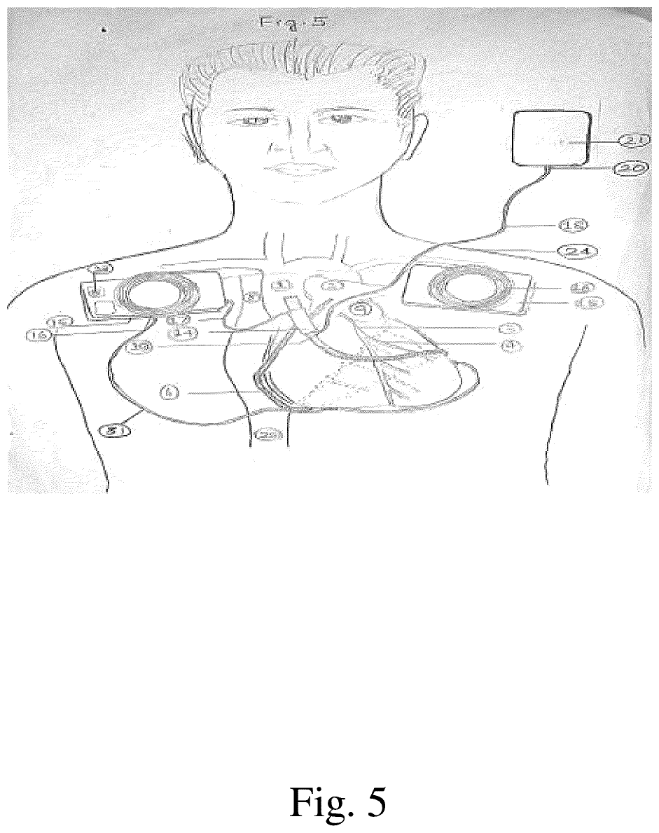

In the preferred embodiment in physiological position, as shown in FIG. 5, the proximal end (11) of the hollow tubular member (10) is anastomosed to any suitable chamber containing oxygen rich blood like the aorta (1), subclavian artery (28), left atrium (9), pulmonary veins (13) etc, and the distal end (12) is anastomosed to a suitable structure like pulmonary artery (2), right atrium, superior vena cava (8), inferior vena cava (25), aorta (1), coronary arteries (3, 4, 5, 6, 7) etc. One or more ‘anastomoses’ are made between the distal portion (beyond the ‘mechanical pump’ (14) assembly) of the body (10) of the tubular member with the coronary arteries (3, 4, 5, 6, 7). The anastomosis between the tubular member body (10) and the Left Anterior Descending artery (3) is labelled as 10A3. Similarly, The anastomosis between the tubular member body (10) and the Diagonal branch (4) of Left Anterior Descending artery is labelled as 10A4, the anastomosis between the tubular member body (10) and the Obtuse marginal branch (5) of the left circumflex artery is labelled as 10A5, the anastomosis between the tubular member body (10) and the Posterior Descending artery (7) is labelled as 10A7 (not illustrated in the diagrams), the anastomosis between the tubular member body (10) and the Right coronary artery (6) is labelled as 10A6, and so on.

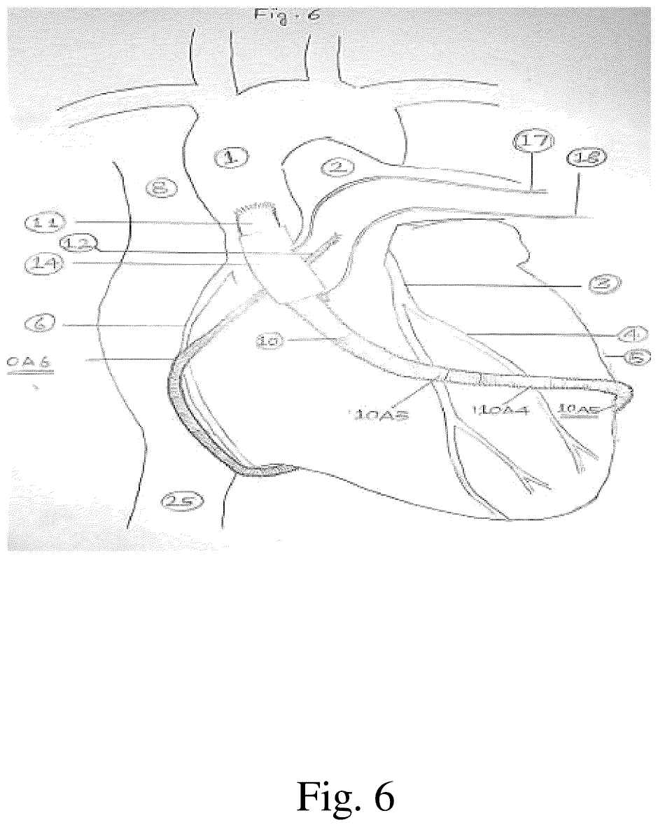

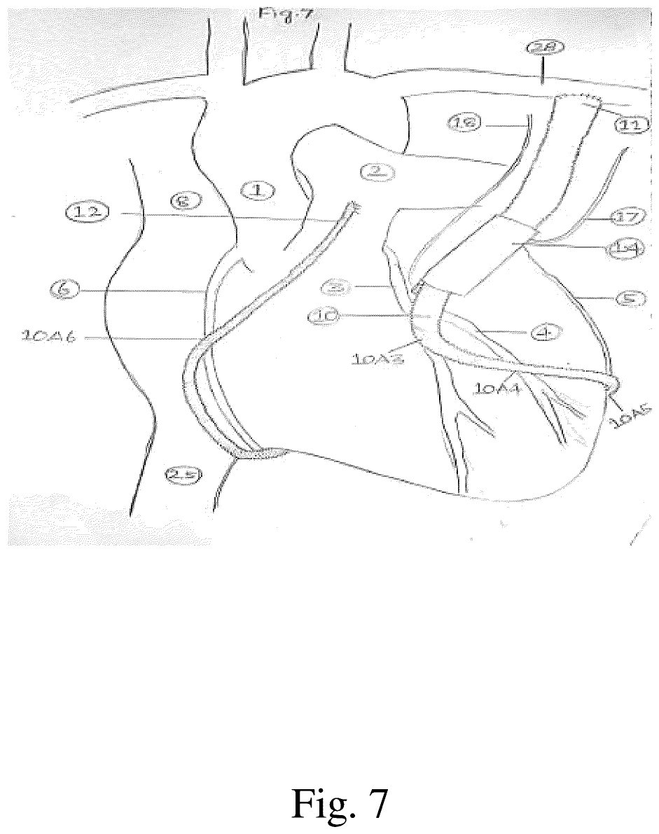

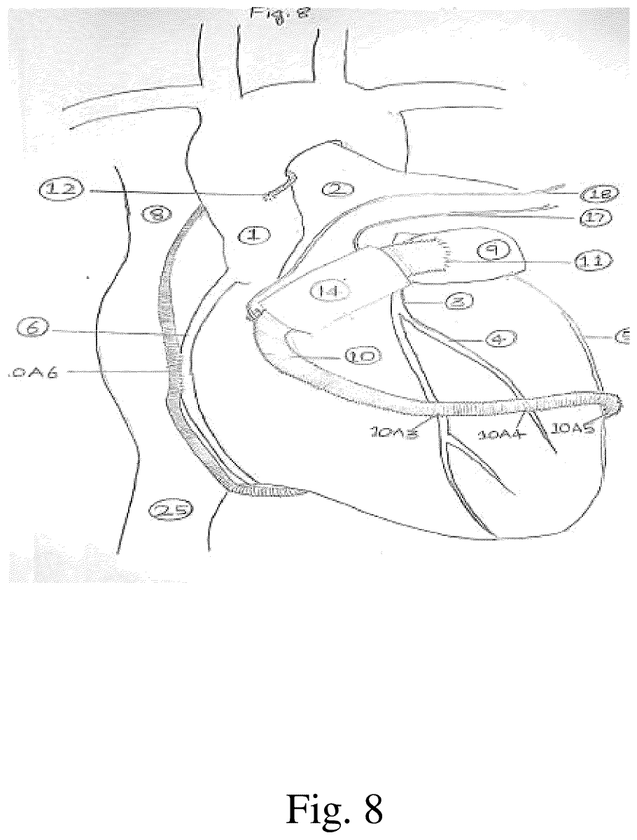

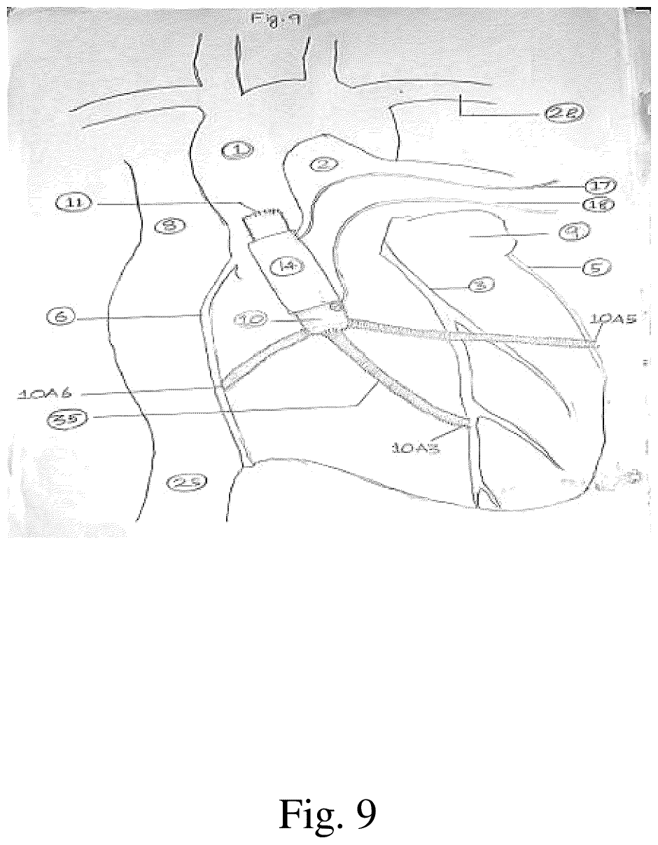

A few anatomical locations of preference for anastomosing the device are shown in FIG. 6, FIG. 7 and FIG. 8. In FIG. 6, the proximal end (11) of the tubular member (10) is anastomosed to the aorta (1) and the distal end is anastomosed to the pulmonary artery (2). In FIG. 7 the proximal end (11) of the tubular member (10) is anastomosed to subclavian artery (28) and the distal end (12) is anastomosed to the pulmonary artery (2). The distal end is anastomosed to a chamber which generally, but not compulsorily, has a lower blood pressure to enable it to work as a pressure release system, particularly during cardiac systole. Choice of the left subclavian artery (28) for proximal anastomosis is helpful for inserting this device in minimally invasive cardiac surgeries (MICS), where the distal end of the tubular member (10) is anastomosed with one of the coronary arteries and the excess length trimmed away. In FIG. 8, the proximal end (11) of the tubular member (10) is anastomosed to the left atrium (9) and the distal end (12) is anastomosed to the aorta (1). In this arrangement too, like in others, the distal tip (12) ends in one of the coronary arteries too, and the excess length is trimmed away. In FIG. 9, there is an alternate embodiment of the tubular member (10), where its distal portion gives away multiple branches (35), (‘distribution channels’), which is anastomosed individually to one or more coronary arteries.

Near to its proximal end (11), the tubular member body (10) houses an electrically driven ‘mechanical pump’ assembly (14). This pump pushes the blood coming from the proximal (11) side of the tubular member, towards the distal (12) side, thereby increasing the blood pressure in the distal portion of the tubular member (10) to a level which is higher than the systemic blood pressure (‘suprasystemic pressure’) at least during diastole, ensuring a high coronary perfusion pressure (CPP) thereby improving coronary perfusion even in failing hearts, despite the lower duration of diastole. This improvement in coronary perfusion is illustrated in FIG. 10. The ‘mechanical pump’ is of any suitable design, including impeller pump, axial pump, centrifugal pump, pistons, pneumatically driven pump or other designs. The electrical energy required to run the pump is provided to it from one or more battery power source (15), implanted in the body. The ‘mechanical pump’ (14) is connected to the battery power source (15) through a power cable (17). In one embodiment, the mechanical pump assembly comprises of a rotor (27) which is surrounded by an electromagnet (26). The rotor spins due to the changes in electromagnetic force created by the electromagnet and due to its movement, the blood is pushed into the coronary arteries through the anastomoses (10A3, 10A4, 10A5, 10A6, 10A7 etc) with suprasystemic pressure, thereby improving the cardiac perfusion. In place of the rotor, any suitable pumping mechanism is substituted to achieve the desired results.

Into the distal portion of the body (10) of the hollow tubular member, beyond the ‘mechanical pump assembly’ (14), a doppler flow sensor (30) is located, which senses the flow velocity in the tubular member. This information is transmitted to the CPU (22), which is used by the CPU to adjust the speed of the ‘mechanical pump’ (14)

The coronary infusion channel (18) is a fine, hollow tubular structure made of a suitable synthetic polymer or silicone material. It has at least one lumen extending from its proximal end to its distal end. The distal end of the coronary infusion channel (18) is positioned into the distal portion of the body (10) of the hollow tubular member preferably beyond the ‘mechanical pump assembly’ (14), through a one-way valve (19). The proximal end (20) of the coronary infusion channel (18) is preferably positioned transcutaneously (outside the body of the patient or animal) or subcutaneously. The proximal end of every lumen of the coronary infusion channel houses a ‘coronary infusion port’ (20) with an in-built one-way valve, which enables infusion of various medicines or combination of medicines without the risk of backflow from the coronary circulation. To this coronary infusion port, a syringe or a suitable mechanical pump (21) is connected to provide intermittent or continuous delivery of one or more of cardiostimulant medications, vasodilators, stem cells, etc, directly into the coronary circulation. The vasodilators administered in this manner is selectively dilates the coronary blood vessels, the antiarrhythmic medicines delivered in this manner stabilises the heart rhythm, the cardiostimulant medicines administered in this manner improves the force of contraction using physiologic energy (against electrical energy as used in other forms of mechanical circulatory support systems), and the stem cells delivered in this manner enables the rejuvenation and recovery of cardiac muscle which had infarcted previously. The target specific delivery of these medicines reduces their required dosage to less than 5% of their usual intravenous dose, thereby reducing their side effects dramatically, tilting the balance towards recovery of cardiac function. One of the lumens of the coronary infusion channel (18) is connected to a transducer to monitor the coronary artery pressure in real time, which is sensed by the CPU (22) to adjust the speed of the pump (14). The coronary infusion channel (18) is used to inject contrast agents to perform fluoroscopic examination of the coronary arteries. After a duration of several weeks to months, after satisfactory recovery of cardiac function, the coronary infusion channel (18) is removed from the device and the body just by pulling its distal end, thereby eliminating the risk of infection.

The mechanical pump (14) is controlled by a central processing unit, CPU, (22), which senses the heart rate, cardiac rhythm and the blood flow in the tubular member. It increases the speed of the mechanical pump (14) if it senses the heart rate to be on the higher side, indicating strenuous physical activity. The CPU is connected to at least one epicardial electrode (31), through which it monitors the heart rate, senses abnormal cardiac rhythms and activates an in-built cardioverter-defibrillator to defibrillate the heart in case of life-threatening cardiac arrhythmias. The CPU (22) is connected to a SIM card (23) and transmits the information wirelessly to several smartphones and other electronic instruments of the patient, his family and healthcare providers, and is controlled wirelessly by the patient and the authorised healthcare personnel through an ‘App’ based interface.

The mechanical pump (14) and the CPU (22) are powered by one or more batteries (15) implanted in the body in suitable locations. These batteries (15) are recharged wirelessly through Transcutaneous Energy Transfer (TET). To achieve TET, receiver coils (16) are connected to receiver circuit (32), which are connected to battery power source (15). These receiver coils (16) are implanted in subcutaneous locations in the body. When transmitter coils (29) which are connected to a transmitter circuit (33) and to an ‘external power source’ (34), are placed immediately across the skin (24) over the site where the receiver coils (16) are implanted subcutaneously, transcutaneous energy transfer (TET) takes place and the battery (15) gets charged. Since this device uses electrical energy only for coronary augmentation and uses physiological energy to improve cardiac output, the electrical energy requirement is just about 10% of Ventricular Assist Devices (VADs) and hence the energy transmitted through TET itself is sufficient, eliminating the need for a transcutaneous drive line, thereby eliminating the risk of life-threatening driveline infection and sepsis.

To conclude, this device and system augments the coronary artery circulation in a manner much more effectively than the prior art. Prior art cardiac assist devices only affect the coronary arteries remotely and indirectly since they assist circulation throughout the whole body. The entire compliance capacity of the peripheral circulation system overcomes in order to benefit the coronary arteries. The present invention perfuses the coronary arteries directly. This device also provides a channel to infuse cardiostimulant medications into coronary circulation, thereby improves cardiac output in out-of-hospital settings, allowing the patients with heart failure to lead a productive life. It gives the heart a chance to recover by providing a port for multiple cycles of stem cell therapy. It also uses minimal electrical power and is self-sufficient on TET, thereby eliminating the need for a driveline its associated inconvenience and life-threatening complications.

Since this device uses electric energy to only improve coronary perfusion and uses physiological energy through inotrope infusion to improve cardiac output (as opposed to ventricular assist devices, which use electric energy to improve cardiac output), the electrical energy used by this device is roughly one tenth of that of ventricular assist devices. Hence this device is easily run on wireless charging and there is no need for a power supply line extending through the patient's skin (percutaneous) which needs to be connected to battery packs round the clock. This design takes away the discomfort of carrying a heavy battery pack round the clock and eliminates the risk of driveline infection which has a very high incidence of life-threatening sepsis. Also, this design allows the patients to engage in contact sports and swimming which were not possible for patients under ventricular assist device (VAD) support.

We claim:

1. A device (100) for improving the cardiac contractility in patients with heart failure to receive real-time data through smartphones comprising:
a. a tubular member body (10) a vascular conduit having a proximal end (11) and a distal end (12) and a Doppler sensor (30);
b. an electrically driven ‘mechanical pump’ (14) is housed in the tubular member body (10) and is connected to an implantable battery power source (15) through a power cable (17);
c. coronary infusion channel (18) comprises one or more lumens wherein at least one lumen extends from its proximal end to its distal end, and
d. a central processing unit (22) connected to the implantable battery power source (15).

2. The device (100) for improving the cardiac contractility in patients with heart failure to receive real-time data through smartphones as claimed in claim 1 wherein the tubular member body (10) is a vascular conduit of about 15-40 cm in length, and is 5-15 mm wide at the proximal end (11) and about 5 mm or less at the distal end (12).

3. The device (100) for improving the cardiac contractility in patients with heart failure to receive real-time data through smartphones as claimed in claim 1 wherein the vascular conduit is made of suitable biocompatible, non-thrombogenic material which is natural or synthetic or a combination of both, in origin.

4. The device (100) for improving the cardiac contractility in patients with heart failure to receive real-time data through smartphones as claimed in claim 1, wherein the mechanical pump (14) is preferably but not limited to axial flow, centrifugal flow, piston or pneumatically driven design.

5. The device (100) for improving the cardiac contractility in patients with heart failure to receive real-time data through smartphones as claimed in claims 1 and 4, wherein the mechanical pump (14) is connected to the implanted battery power source (15) through a power cable (17) and is powered by the battery power source (15)

6. The device (100) for improving the cardiac contractility in patients with heart failure to receive real-time data through smartphones as claimed in claims 1 and 4, wherein the mechanical pump (14) maintains suprasystemic blood pressure inside the inner lumen of the tubular member body (10) at least during diastole.

7. The device (100) for improving the cardiac contractility in patients with heart failure to receive real-time data through smartphones as claimed in claims 1 and 4, wherein the coronary infusion channel (18) is made of synthetic polymer selected from plastic or silicone.

8. The device (100) for improving the cardiac contractility in patients with heart failure to receive real-time data through smartphones as claimed in claims 1 and 4, wherein the implantable battery power source (15) supplies electrical energy to the mechanical pump (14) and the central processing unit ‘CPU’ (22)

9. The device (100) for improving the cardiac contractility in patients with heart failure to receive real-time data through smartphones as claimed in claim 1 and 8, wherein the battery power source (15) is connected to one or more receiver coils (16).

10. The device (100) for improving the cardiac contractility in patients with heart failure to receive real-time data through smartphones as claimed in claims 1 and 9, wherein the one or more receiver coils (16) are placed subcutaneously in at least one location to enable wireless charging of the implanted battery power source (15), by placement of transmitter coils (29) directly over them across the skin (24).

11. The device (100) for improving the cardiac contractility in patients with heart failure to receive real-time data through smartphones as claimed in claim 1, wherein the central processing unit (22) monitors the vital parameters such as heart rate, heart rhythm using at least one epicardial electrode (31), blood flow velocity and blood pressure through the Doppler sensor (30) and controls the speed of mechanical pump (14).

12. The device (100) for improving the cardiac contractility in patients with heart failure to receive real-time data through smartphones as claimed in claim 1, wherein the central processing unit (22) has an in-built SIM card (23) and can be connected to electronic devices to transmit the vital parameters in real time.

13. The device (100) for improving the cardiac contractility in patients with heart failure to receive real-time data through smartphones as claimed in claim 1, wherein the proximal end of the coronary infusion channel (18) lies outside the body of human or animal or buried beneath the skin of human or animal and houses at least one coronary infusion port (20) with an in-built one-way valve; and wherein the distal end of the coronary infusion channel opens into the inner lumen of the distal portion of the hollow tubular member body (10), just distal to the mechanical pump (14) assembly, through a one-way valve (19).

14. The device (100) for improving the cardiac contractility in patients with heart failure to receive real-time data through smartphones as claimed in claims 1 and 13, wherein the coronary infusion port (20) is further attached with a coronary infuser pump (21).

15. The device (100) for improving the cardiac contractility in patients with heart failure to receive real-time data through smartphones as claimed in claims 1 and 14, wherein the coronary infuser pump (21) is loaded with a combination of cardio stimulant medicines and other molecules.

16. The device (100) for improving the cardiac contractility in patients with heart failure to receive real-time data through smartphones as claimed in claim 1, wherein the central processing unit (22) is an electronic device which controls the mechanical pump (14)

17. The device (100) for improving the cardiac contractility in patients with heart failure using IOT technology to receive real-time data through smartphones as claimed in claim 16, wherein the central processing unit (22) incorporates a cardioverter-defibrillator which works using the epicardial electrodes (31).

18. The device (100) for improving the cardiac contractility in patients with heart failure to receive real-time data through smartphones as claimed in claim 1, wherein the proximal end (11) of the tubular member body (10) is connected with the lumen of a vessel or chamber containing oxygen rich blood such as aorta (1), subclavian artery (28), left atrium (9) or one or more pulmonary veins (13); wherein the distal portion of the tubular member body (10) beyond the mechanical pump (14) assembly, is connected in a fluidly communicating manner (‘anastomosed’) with at least one coronary artery (10A3, 10A4, 10A5, 10A6, 10A7) either directly or through distribution channels (35); wherein the distal end (12) of the tubular member is connected to any one of the vessel selected from coronary artery (3, 4, 5, 6, 7), pulmonary artery (2), atria, aorta (1), vena cavae (9, 25) or a suitable chamber, to ensure pressure release, particularly during systole.

19. The device (100) for improving the cardiac contractility in patients with heart failure to receive real-time data, through smartphones as claimed in claim 1, wherein the said Central Processing Unit (CPU) monitors the heart rate with using the implanted ECG lead and controls the speed in which the pump rotor is spinning, thereby delivering more coronary blood during exertion.

20. The device (100) for improving the cardiac contractility in patients with heart failure to receive real-time data, through smartphones as claimed in claim 1, wherein the said CPU has an in-built SIM card, and is connected to multiple smartphones to transmit vital data including the pump speed, coronary artery pressure, heart rate, percentage of power remaining in the battery etc.

21. The device (100) for improving the cardiac contractility in patients with heart failure using IOT technology to receive real-time data through smartphones as claimed in claim 1, wherein the said Coronary pressure transducer is used to gauge pressure in the coronary channels.

22. A method for improving cardiac function by improving coronary perfusion pressure by implanting the device as claimed in claim 1, wherein the proximal end (11) of the tubular member body (10) is connected with the lumen of a vessel or chamber containing oxygen rich blood such as aorta (1), subclavian artery (28), left atrium (9) or one or more pulmonary veins (13); wherein the distal portion of the tubular member body (10) beyond the mechanical pump (14) assembly, is connected in a fluidly communicating manner (‘anastomosed’) with at least one coronary artery (10A3, 10A4, 10A5, 10A6, 10A7) either directly or through coronary distribution channels (35);
wherein the distal end (12) of the tubular member is connected to any one of the vessel selected from coronary artery (3, 4, 5, 6, 7), pulmonary artery (2), or its branches atria, aorta (1), vena cavae (9,25) or suitable chamber, to ensure pressure release, particularly during systole; and by physiologically improving the cardiac pumping ability and regeneration of cardiomyocytes by direct infusion of cardiostimulant medicines and stem cells through the coronary infusion channel (18).
